(19) United States
(12) Patent Application Publication
(10) Pub. No.: US 2023/0407231 A1
(43) Pub. Date: Dec. 21, 2023

(54) AUTOMATED APPARATUS FOR EFFECTIVELY ENRICHING NEUROGENIC EXOSOMES IN BLOOD

(71) Applicant: Beijing Kaixiang Hongkang Biotechnology Co., LTD, Beijing (CN)

(72) Inventors: Yongxiang MA, Beijing (CN); Jia LI, Beijing (CN); Xiaojing PANG, Beijing (CN)

(21) Appl. No.: 18/363,722

(22) Filed: Aug. 1, 2023

Related U.S. Application Data
(63) Continuation-in-part of application No. PCT/CN2022/079045, filed on Mar. 3, 2022.

(30) Foreign Application Priority Data
Mar. 5, 2021 (CN) 202110247374.3
Nov. 1, 2022 (CN) 202211354035.6
Nov. 1, 2022 (CN) 202211366223.0

(51) Int. Cl. C12M 1/36; C12M 1/00; C12M 1/26

(52) U.S. Cl. CPC C12M 41/48; C12M 33/10; C12M 41/14

(57) ABSTRACT

An automated apparatus for enriching the neurogenic exosomes in blood has a reaction frame at a bottom and a movable operation frame above the reaction frame, and a control structure for controlling the operation frame to run according to a preset track and controlling the reaction frame to react according to a set program. The reaction frame has an oscillation incubation structure, a centrifugal structure, a reagent placing structure, a consumable placing structure, a waste placing structure, a sample placing structure, a sample information identification structure, and a post-reaction enriched sample collection structure. The operation frame has a tube body moving structure for moving a tube body among different structures and a liquid taking and adding structure for transferring liquid in the whole process. The reaction frame or the operation frame has an EP tube marking and identification structure used when an EP tube is used for the first time.

### TECHNICAL FIELD

The present invention belongs to the technical field of medical devices and particularly relates to a device for automatically enriching exosomes. More specifically, it relates to an automated apparatus for effectively enriching neurogenic exosomes in blood.

### BACKGROUND

Neurodegenerative diseases are caused by loss of neurons and/or their myelin sheaths, which get worse over time and lead to dysfunction. These neurodegenerative diseases include Alzheimer's disease (AD), Parkinson's disease (PD), and Prion disease. However, at present, the definite clinical diagnosis of neurodegenerative diseases depends on positron emission tomography (PET), and the diseases often progress to an untreatable and irreversible stage when pathological changes occur.

Diagnosis of these diseases can be advanced by detecting related biomarkers through cerebrospinal fluid. However, sampling cerebrospinal fluid causes severe trauma to the human body, and the patient willingness is low. Few patients would undertake diagnosis in advance before any symptoms show, and in most cases, this kind of detection is performed after symptoms occur. Moreover, collecting cerebrospinal fluid is very difficult and highly risky, which puts a heavy demand on the operator's capacity. All these factors make it hard to become a conventional method for detecting the risk of the diseases.

Therefore, it is necessary to seek out a method that facilitates collection and detection, and preferably can be included in regular annual physical examination for screening. With the intensive research of exosomes, it has been found that neurogenic exosomes in blood can be used as a sample to detect relevant indexes of neurodegenerative diseases, so that the diagnosis of the diseases can be greatly advanced, and the subject has higher compliance to the sample than to cerebrospinal fluid.

If an automated apparatus is available to complete the above process, efficiently enrich neurogenic exosomes from a subject, and detect the relevant information, it would be possible to find out if the subject is at the risk of the disease in advance, and conduct early prevention. Through the promotion and application of such detection mode, excessive neurodegenerative diseases occurring due to the aging of the population can be greatly reduced.

In view of the problem that there is no automated apparatus for effectively and rapidly enriching neurogenic exosomes in blood in the prior art, the present invention provides an automated apparatus for effectively enriching neurogenic exosomes in blood.

### SUMMARY

Neurogenic exosomes in blood can be obtained only by enrichment. At present, the enrichment of common exosomes is mostly completed manually. However, the manual operation is slow and prone to errors.

After extensive trials, the team of inventors has found that the process of enriching neurogenic exosome involves multiple steps, including cooling, centrifugation, oscillation, sample loading, pipetting, which is time-consuming and requires operators to keep track of time. It consumes a great deal of manpower and time during the whole process. Moreover, incorrect conclusions are likely to be made from pure manual operation due to errors in samples, and it is completely impossible to achieve medical examinations for a large sample size since manual operations are not feasible for scaling up. However, after extensive trials, it has been found that the core steps of neurogenic exosome enrichment are the multiple transitions between the oscillation incubation operation and the centrifugation operation. Therefore, the steps are incorporated in an automated apparatus for enriching exosomes.

Disclosed is an automated apparatus for effectively enriching neurogenic exosomes in blood, comprising a reaction frame at the bottom and a movable operation frame above the reaction frame, and a control structure for controlling the operation frame to run according to a preset track and controlling the reaction frame to react according to a set program, wherein the reaction frame is provided with an oscillation incubation structure, a centrifugal structure, a reagent placing structure, a consumable placing structure, a waste placing structure, a sample placing structure, a sample information identification structure, and a post-reaction enriched sample collection structure, all necessary for realizing exosome enrichment; the operation frame is provided with a tube body moving structure for moving a tube body among different structures and a liquid taking and adding structure for transferring liquid in the whole process; in addition, the reaction frame or the operation frame is provided with an EP tube marking and identification structure used when an EP tube is used for the first time. The apparatus further comprises a storage module capable of storing information, and the control structure controls the oscillation rotating speed and the amplitude of the oscillation incubation structure and controls the temperature required by the corresponding step of the oscillation incubation structure; the control structure controls and monitors the reagent amount of the reagent placing structure, and gives a prompt for replacement when no reagent is left; the control structure also controls centrifugal parameters of the centrifugal structure; the control structure monitors and records information of samples in a corresponding placement hole of the sample placing structure; the control structure controls the sample information identification structure 17 to identify sample tube information and stores the sample tube information in the storage module; the control structure controls a liquid taking and adding tube to move to carry out liquid taking and adding processes, the control structure controls the tube body moving structure to grab a corresponding tube body to move to a corresponding area of a next corresponding structure, and the control structure controls the EP tube marking and identification structure to scan or identify information on the EP tube and matches the information with the corresponding sample tube information, so as to avoid confusion or loss of patient information in the moving process; the control structure controls the final EP tube to be provided in the enriched sample collection structure.

In specific operations, the control structure controls the information identification structure to firstly identify information codes of a sample tube and store the information codes into a storage unit of the apparatus; after identification, the sample tube is sequentially set in the sample placing structure. Then, the tube body moving structure on the operation frame grabs the sample tubes in the sample placing structure and transfer it to the next processing structure, i.e., oscillation incubation structure according to a set sequence; after the sample tubes enter the oscillation incubation structure, the liquid taking and adding structure moves to the consumable placing structure according to control setting, mounts corresponding consumables, and then moves to the reagent placing structure; after obtaining corresponding reagents, the liquid taking and adding structure moves to the position where the corresponding sample tube is placed in the oscillation incubation structure to add the reagent; following the addition of the reagent, the liquid taking and adding structure moves away to the waste placing structure and discard the waste consumables in the waste placing structure. After the reagent is added, the control structure controls the oscillation incubation structure to conduct oscillation or incubation, and during the oscillation incubation process, any steps requiring reagent addition follow the foregoing procedure. When it is required to move a sample tube into a centrifugal structure, the control structure controls the tube body moving structure to grab the sample tube and place the sample tube into the centrifugal structure for centrifugation; if liquid needs to be added after centrifugation, the reagent is added according to the foregoing steps of liquid taking and adding; when it is required to reserve the supernatant and transfer the supernatant into the EP tube, the EP tube is first placed into the structure at the next step, the EP tube marking and identification structure scans or identifies the information on the EP tube and matches the information with the corresponding sample tube information, and then the liquid in the sample tube is transferred into the corresponding EP tube by using the liquid taking and adding structure; when the EP tube is in the oscillation incubation structure, oscillation incubation is conducted according to an oscillation incubation process, and the reagent is added when required; when the sample tube needs to be centrifuged again, the sample tube is moved into the centrifugal structure using the tube body moving structure. When it is required to reserve the precipitate after the centrifugation is finished, the control structure controls the liquid taking and adding structure to extract the supernatant from the EP tube and discard the supernatant in the waste placing structure. After enrichment is completed under the control of the control structure according to the step of exosome enrichment, the EP tube obtained after enrichment is sealed and placed in the enriched sample collection structure.

Further, it is required to provide a plurality of oscillation incubation structures, centrifugal structures, reagent placing structures, consumable placing structures, and waste placing structures according to the operation steps. In this way, the moving distance can be reduced for the operation frame at the top, and the continuity of the whole automatic process is improved.

Further, when a sample or EP tube is at the stage of the oscillation incubation, in case the step of adding a reagent is required, a corresponding reagent placing structure, a consumable placing structure, and a waste placing structure are provided in the vicinity of the corresponding oscillation incubation structure. This facilitates convenient access to the reagent and nearby disposal.

Or, in the case only one batch of samples are enriched for one operation, and the sample tube has a structure and a shape the same as the structure and shape of the EP tube, 1 oscillation incubation structure and 1 centrifugal structure are provided in the whole process. In this way, the whole apparatus has a simple structure and occupies less space, and such apparatus is mainly targeted at the enrichment process of relatively small samples and can thus be used in laboratory.

Or, in the case only one batch of samples are enriched for one operation, and the sample tube has a structure and a shape different from the structure and shape of the EP tube, a minimum number of oscillation incubation structures and centrifugal structures are provided according to the requirements for tubes in different shapes. In this way, the whole apparatus only enriches one batch of samples in one process.

Further, in the case only one batch of samples are enriched for one process, one reagent placing structure, one consumable placing structure, and one waste placing structure are provided, and all the reagent placing structure, the consumable placing structure, and the waste placing structure for exosome enrichment are respectively and uniformly provided. In this way, the space occupied by the apparatus can be further reduced, and it takes less time to move the operation frame since the overall structure is small.

Further, the operation frame is provided on an arrangement top; the arrangement top is provided above the reaction frame and maintains a fixed position relative to the reaction frame, and the arrangement top is provided with a moving track allowing the operation frame to move above any structure and perform a corresponding operation.

Further, the operation frame is an integral frame, and the liquid taking and adding structure and the tube body moving structure are provided thereon; the liquid taking and adding structure and the tube body moving structure are capable of moving up and down on the operation frame, allowing a single structure to perform all functions.

Or, two operation frames are provided, namely a liquid taking and adding operation frame and a tube body moving operation frame; the operation frames move to the positions above the corresponding structures respectively for performing an action, and when one operation frame is in action, the other operation frame moves along a moving track to a position where the other operation frame will not be interfered with.

Further, the liquid taking and adding structure is provided with the same number of operating heads as the tube body moving structure, which can ensure that the same number of sample tubes are clamped and corresponding reagents are added.

Further, corresponding to a single type of reagent, the reagent placing structure is provided with liquid taking ports in a quantity equal to the number of the operating heads of the liquid taking and adding structure, and the positional relationship of the liquid taking ports corresponds to the positional relationship of the operating heads; corresponding to a single type of consumable, the consumable placing structure is provided with consumable taking ports in a quantity equal to the number of the operating heads of the liquid taking and adding structure, and the positional relationship of the consumable taking ports corresponds to the positional relationship of the operating heads.

Further, the number of single-row placement holes is an integral multiple of the number of operating heads; or, the number of columns of the placement holes matches the number of the operating heads, so that the sample tubes can be obtained according to a proper sequence.

Further, a fault alarm structure is further provided between each structure and the control structure, and when the corresponding fault alarm structure sends an alarm signal, it indicates a malfunction in the corresponding structure. Or, the control structure is further connected to a display structure, and the display structure displays the alarms from the corresponding alarm structure, prompting maintenance for the corresponding structure malfunction. This arrangement ensures that the alarm is given in a timely manner when some problems occur in this complex structure.

Further, the number of reactions corresponding to the amount of any two different reagents in the reagent placing structure is equal, and when the number of reactions reaches a certain threshold, a prompt or display on the display structure indicates the need for replacement.

Further, the number of reactions corresponding to the amount of any two different consumables in the consumable placing structure is also equal, or the consumable amount of each consumable is equal to the number of reactions of the reagent, so as to facilitate overall replacement of reagents and consumables.

Further, the waste placing structure is divided into a consumable discarding portion and a liquid discarding portion, handling waste consumables and liquid, respectively.

Further, the capacity of the waste placing structure for accommodating the waste is equal to the space required for accommodating reagents and consumables of one replacement, allowing it to be replaced together with the reagents and consumables. This ultimately reduces monitoring difficulties and increases efficiency.

Further, the reagent placing structure, the consumable placing structure and the waste placing structure are provided under the arrangement plate and in the middle of the reaction frame, and are provided under the arrangement plate in a combined manner, so that replacement is facilitated.

Further, the EP tube marking and identification structure is provided on the tube body moving structure, and distinct identification structures are provided on the top of each EP tube, allowing for one-to-one correspondence. The EP tube is scanned and identified from the above and matched with the corresponding sample tube information, so that the purpose of information matching is achieved, and the process is simple and convenient.

Further, the entire enrichment process comprises a plurality of oscillation incubation and centrifugation processes, each process comprising an oscillation incubation and centrifugation step; the reaction frame of the whole apparatus is divided into a plurality of corresponding reaction areas according to the times of the oscillation incubation and centrifugation process. Further, each corresponding area is provided with an oscillation incubation structure, centrifugal structure, reagent placing structure, consumable placing structure, and waste placing structure.

Further, a set of operation frame with a liquid taking and adding structure and a tube body moving structure is provided above each reaction area. In this way, a systematic process of liquid taking and tube body clamping can be realized without any conflicts in usage.

Further, the moving track of the operation frame is a track for facilitating movement of the operation frame for liquid taking and tube body clamping.

Further, the moving track is also divided into different areas to allow the moving track to move in the current area and take samples or EP tubes in an up-moving area.

Further, the enriched sample collection structure is provided with an EP tube consumable placing structure, or the EP tube consumable placing structure is provided in another nearest reaction area; when proceeding to last step, the EP tube is moved to the enriched sample collection structure zone through the tube body moving structure, then the supernatant obtained after enrichment is transferred to the EP tube through the liquid taking and adding structure for collection, and then detection is performed.

Disclosed is the use of an automated apparatus for effectively enriching neurogenic exosomes in blood in enriching exosomes in blood.

Compared with devices in the prior art, the technical solutions of the present invention can well solve the problem of exosome enrichment in the prior art, and can complete exosome enrichment automatically, so that exosome detection can be well promoted. If promoted properly in the future, it could become a means for early detection of neurogenic exosomes in blood, facilitating early diagnosis and intervention in neurodegenerative diseases. This would help alleviate the burden on society due to the increasing occurrence of neurodegenerative diseases in the aging population in China.

### BRIEF DESCRIPTION OF THE DRAWINGS

FIG. 1 is a schematic diagram of the overall structure according to the present invention.

FIG. 2 is a schematic top view of the structure of the reaction frame according to the present invention.

FIG. 3 is a schematic side view of the structure of a part of the operation frame according to the present invention.

FIG. 4 is a schematic diagram of the overall structure comprising only one reaction area according to the present invention.

FIG. 5 is a schematic structural diagram of the working flow of the apparatus according to the present invention.

FIG. 6 is a schematic diagram of the modular structures of the apparatus according to the present invention.

FIG. 7 is a centrifugal recognition system in the exosomes enrichment system of the present invention;

FIG. 8 is a schematic diagram of the structure of a centrifugal component in the exosomes enrichment system of the present invention;

FIG. 9 is a top view of the centrifugal component in the exosomes enrichment system of the present invention;

FIG. 10 is a schematic diagram of the internal structure of the centrifugal component in the exosomes enrichment system of the present invention.

FIG. 11 is a schematic diagram of the internal structure of the centrifugal component in the exosomes enrichment system of the present invention.

FIG. 12 is a schematic diagram of the structure of a centrifuge tube in the exosomes enrichment system of the present invention.

FIG. 13 is a schematic diagram of the lateral structure of a centrifugal instrument controller for the exosomes enrichment system of the present invention.

FIG. 14 is a schematic diagram of the front view structure of a color sensor for the exosomes enrichment system of the present invention

FIG. 15 is a schematic diagram of the structure of a centrifugal component exposed to the centrifugal instrument of the present invention

In the figures, 1 represents a reaction frame; 11 represents an oscillation incubation structure; 12 represents a centrifugal structure; 13 represents a reagent placing structure; 14 represents a consumable placing structure; 15 represents a waste placing structure; 151 represents a consumable discarding portion; 152 represents a liquid discarding portion; 16 represents a sample placing structure; 17 represents a sample information identification structure; 18 represents an enriched sample collection structure; 2 represents an operation frame; 21 represents a tube body moving structure; 22 represents a liquid taking and adding structure; 23 represents an EP tube marking and identification structure; 24 represents an arrangement top; 25 represents an operating head; 26 represents a moving track; 3 represents a control structure; 4 represents a storage module; 5 represents a fault alarm structure;
212 centrifuge chamber; 213 sampling base; 214 hole site; 221 dust cover; 222 cover; 231 mechanical arm; 232 clamping jaw; 241 color identification sensor; 251 identification structure; 261 centrifuge tube; 311 controller; 321 infrared sensor; 3211 emitting component; 3212 receiving component; 322 light shielding plate.

### DETAILED DESCRIPTION

### Example 1

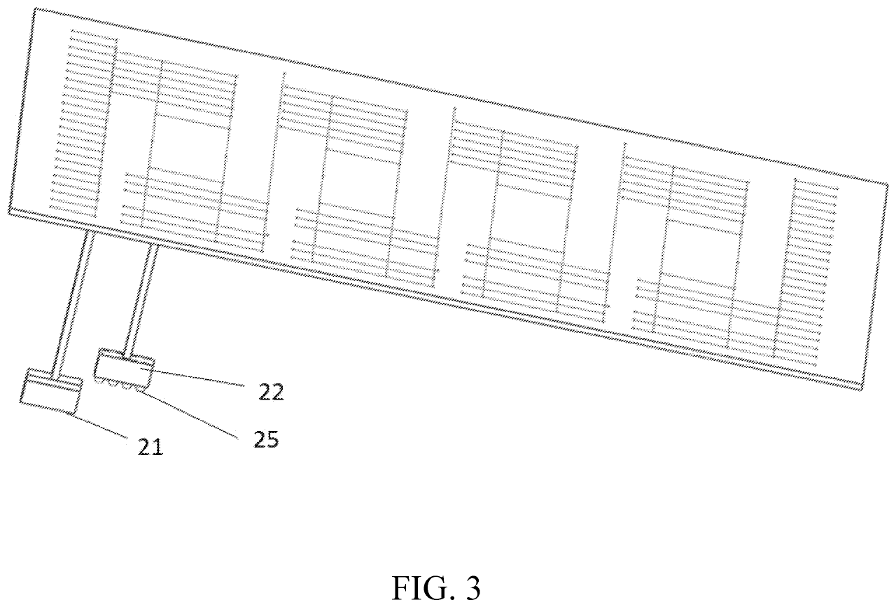

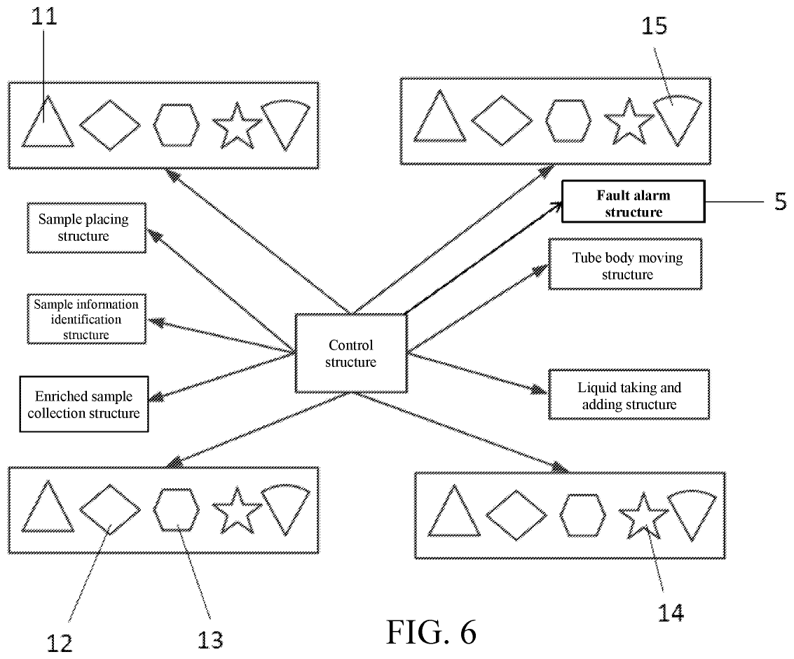

Referring to FIGS. 3-4 and 6, disclosed is an automated apparatus for effectively enriching neurogenic exosomes in blood, comprising a reaction frame 1 at the bottom and a movable operation frame 2 above the reaction frame, and a control structure 3 for controlling the operation frame 2 to run according to a preset track and controlling the reaction frame 1 to react according to a set program, wherein the reaction frame 1 is provided with an oscillation incubation structure 11, a centrifugal structure 12, a reagent placing structure 13, a consumable placing structure 14, a waste placing structure 15, a sample placing structure 16, a sample information identification structure 17, and a post-reaction enriched sample collection structure 18, all necessary for realizing exosome enrichment; the operation frame 2 is provided with a tube body moving structure 21 for moving a tube body among different structures and a liquid taking and adding structure 22 for transferring liquid in the whole process; in addition, the reaction frame 1 or the operation frame 2 is provided with an EP tube marking and identification structure 23 used when an EP tube is used for the first time. The apparatus further comprises a storage module 4 capable of storing information, and the control structure 3 controls the oscillation rotating speed and the amplitude of the oscillation incubation structure 11 and controls the temperature required by the corresponding step of the oscillation incubation structure 11; the control structure 3 controls and monitors the reagent amount of the reagent placing structure 13, and gives a prompt for replacement when no reagent is left; the control structure 3 also controls centrifugal parameters of the centrifugal structure 12; the control structure 3 monitors and records information of samples in a corresponding placement hole of the sample placing structure 16; the control structure 3 controls the sample information identification structure 17 to identify sample tube information and stores the sample tube information in the storage module 4; the control structure 3 controls a liquid taking and adding tube to move to carry out liquid taking and adding processes, the control structure 3 controls the tube body moving structure 21 to grab a corresponding tube body to move to a corresponding area of a next corresponding structure, and the control structure 3 controls the EP tube marking and identification structure 23 to scan or identify information on the EP tube and matches the information with the corresponding sample tube information, so as to avoid confusion or loss of patient information in the moving process; the control structure 3 controls the final EP tube to be provided in the enriched sample collection structure 18.

In specific operations, the control structure 3 controls the information identification structure to firstly identify information codes of a sample tube and store the information codes into a storage unit of the apparatus; after identification, the sample tube is sequentially set in the sample placing structure 16. Then, the tube body moving structure 21 on the operation frame 2 grabs the sample tubes in the sample placing structure 16 and transfer it to the next processing structure, i.e., oscillation incubation structure 11 according to a set sequence; after the sample tubes enter the oscillation incubation structure 11, the liquid taking and adding structure 22 moves to the consumable placing structure 14 according to control setting, mounts corresponding consumables, and then moves to the reagent placing structure 13; after obtaining corresponding reagents, the liquid taking and adding structure moves to the position where the corresponding sample tube is placed in the oscillation incubation structure 11 to add the reagent; following the addition of the reagent, the liquid taking and adding structure 22 moves away to the waste placing structure 15 and discard the waste consumables in the waste placing structure 15. After the reagent is added, the control structure 3 controls the oscillation incubation structure 11 to conduct oscillation or incubation, and during the oscillation incubation process, any steps requiring reagent addition follow the foregoing procedure. When it is required to move a sample tube into a centrifugal structure 12, the control structure 3 controls the tube body moving structure 21 to grab the sample tube and place the sample tube into the centrifugal structure 12 for centrifugation; if liquid needs to be added after centrifugation, the reagent is added according to the foregoing steps of liquid taking and adding; when it is required to reserve the supernatant and transfer the supernatant into the EP tube, the EP tube is first placed into the structure at the next step, the EP tube marking and identification structure 23 scans or identifies the information on the EP tube and matches the information with the corresponding sample tube information, and then the liquid in the sample tube is transferred into the corresponding EP tube by using the liquid taking and adding structure 22; when the EP tube is in the oscillation incubation structure 11, oscillation incubation is conducted according to an oscillation incubation process, and the reagent is added when required; when the sample tube needs to be centrifuged again, the sample tube is moved into the centrifugal structure 12 using the tube body moving structure 21. When it is required to reserve the precipitate after the centrifugation is finished, the control structure 3 controls the liquid taking and adding structure 22 to extract the supernatant from the EP tube and discard the supernatant in the waste placing structure 15. After enrichment is completed under the control of the control structure 3 according to the step of exosome enrichment, the EP tube obtained after enrichment is sealed and placed in the enriched sample collection structure 18.

When a sample or EP tube is at the stage of the oscillation incubation structure 11, in case the step of adding a reagent is required, a corresponding reagent placing structure 13, a consumable placing structure 14, and a waste placing structure 15 are provided in the vicinity of the corresponding oscillation incubation structure 11. This facilitates convenient access to the reagent and nearby disposal.

In the case only one batch of samples are enriched for one operation, and the sample tube has a structure and a shape the same as the structure and shape of the EP tube, 1 oscillation incubation structure 11 and 1 centrifugal structure 12 are provided in the whole process. In this way, the whole apparatus has a simple structure and occupies less space, and such apparatus is mainly targeted at the enrichment process of relatively small samples and can thus be used in laboratory.

In the case only one batch of samples are enriched for one process, one reagent placing structure 13, one consumable placing structure 14, and one waste placing structure 15 are provided, and all the reagent placing structure, the consumable placing structure, and the waste placing structure for exosome enrichment are respectively and uniformly provided. In this way, the space occupied by the apparatus can be further reduced, and it takes less time to move the operation frame 2 since the overall structure is small.

The embodiment of operation frame is as follows: The operation frame 2 is provided on an arrangement top 24; the arrangement top 24 is provided above the reaction frame 1 and maintains a fixed position relative to the reaction frame 1, and the arrangement top 24 is provided with a moving track 26 allowing the operation frame 2 to move above any structure and perform a corresponding operation.

The operation frame 2 is an integral frame, and the liquid taking and adding structure 22 and the tube body moving structure 21 are provided thereon; the liquid taking and adding structure 22 and the tube body moving structure 21 are structures capable of moving up and down on the operation frame 2, allowing a single structure to perform all functions.

The liquid taking and adding structure 22 is provided with the same number of operating heads 25 as the tube body moving structure 21, which can ensure that the same number of sample tubes are clamped and corresponding reagents are added.

Corresponding to a single type of reagent, the reagent placing structure 13 is provided with liquid taking ports in a quantity equal to the number of the operating heads 25 of the liquid taking and adding structure 22, and the positional relationship of the liquid taking ports corresponds to the positional relationship of the operating heads 25; corresponding to a single type of consumable, the consumable placing structure 14 is provided with consumable taking ports in a quantity equal to the number of the operating heads 25 of the liquid taking and adding structure 22, and the positional relationship of the consumable taking ports corresponds to the positional relationship of the operating heads 25.

The number of single-row placement holes is an integral multiple of the number of operating heads 25; or, the number of columns of the placement holes matches the number of the operating heads so that the sample tubes can be obtained according to a proper sequence.

The reagent placing structure 13, the consumable placing structure 14, and the waste placing structure 15 are provided under the arrangement plate and in the middle of the reaction frame 1, and are provided under the arrangement plate in a combined manner, so that replacement is facilitated.

The EP tube marking and identification structure 23 is provided on the tube body moving structure 21, and distinct identification structures are provided on the top of each EP tube, allowing for one-to-one correspondence. The EP tube is scanned and identified from the above and matched with the corresponding sample tube information, so that the purpose of information matching is achieved, and the process is simple and convenient.

The enriched sample collection structure 18 is provided with an EP tube consumable placing structure 14; when proceeding to last step, the EP tube is moved to the enriched sample collection structure 18 zone through the tube body moving structure 21, then the supernatant obtained after enrichment is transferred to the EP tube through the liquid taking and adding structure 22 for collection, and then detection is performed.

### Example 2

On the basis of Example 1, the following embodiments are adjusted:

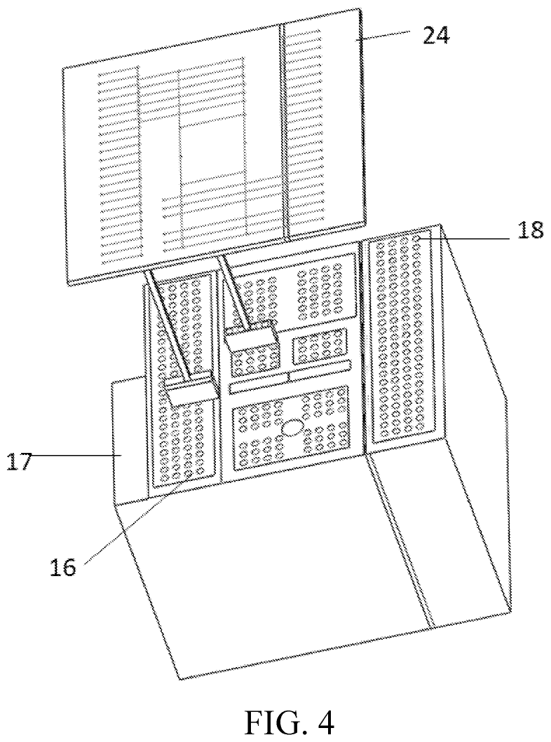

Referring to FIG. 4, in the case only one batch of samples are enriched for one operation, an embodiment where the sample tube has a structure and a shape different from the structure and shape of the EP tube is added, a minimum number of oscillation incubation structures 11 and centrifugal structures 12 are provided according to the requirements for tubes in different shapes. In this way, the whole apparatus only enriches one batch of samples in one process. In the case only one batch of samples are enriched for one process, one reagent placing structure 13, one consumable placing structure 14, and one waste placing structure 15 are provided, and all the reagent placing structure, the consumable placing structure, and the waste placing structure for exosome enrichment are respectively and uniformly provided. In this way, the space occupied by the apparatus can be further reduced, and it takes less time to move the operation frame 2 since the overall structure is small.

An embodiment where 2 operation frames are provided is added. Two operation frames 2 are provided, namely a liquid taking and adding operation frame 2 and a tube body moving operation frame 2; the operation frames move to the positions above the corresponding structures respectively for performing an action, and when one operation frame 2 is in action, the other operation frame 2 moves along a moving track 26 to a position where the other operation frame 2 will not be interfered with.

In order to ensure the use safety, the following embodiment is added: A fault alarm structure 5 is further provided between each structure and the control structure 3, and when the corresponding fault alarm structure 5 sends an alarm signal, it indicates a malfunction in the corresponding structure. Or, the control structure 3 is further connected to a display structure, and the display structure displays the alarms from the corresponding alarm structure, prompting maintenance for the corresponding structure malfunction. This arrangement ensures that the alarm is given in a timely manner when some problems occur in this complex structure.

The number of reagents in the reagent placing structure is limited. Specifically, the number of reactions corresponding to the amount of any two different reagents in the reagent placing structure 13 is equal, and when the number of reactions reaches a certain threshold, a prompt or display on the display structure indicates the need for replacement. The number of reactions corresponding to the amount of any two different consumables in the consumable placing structure 14 is also equal, or the consumable amount of each consumable is equal to the number of reactions of the reagent, so as to facilitate overall replacement of reagents and consumables. The waste placing structure 15 is divided into a consumable discarding portion 151 and a liquid discarding portion 152, handling waste consumables and liquid, respectively. The capacity of the waste placing structure 15 for accommodating the waste is equal to the space required for accommodating reagents and consumables of one replacement, allowing it to be replaced together with the reagents and consumables. This ultimately reduces monitoring difficulties and increases efficiency.

### Example 3

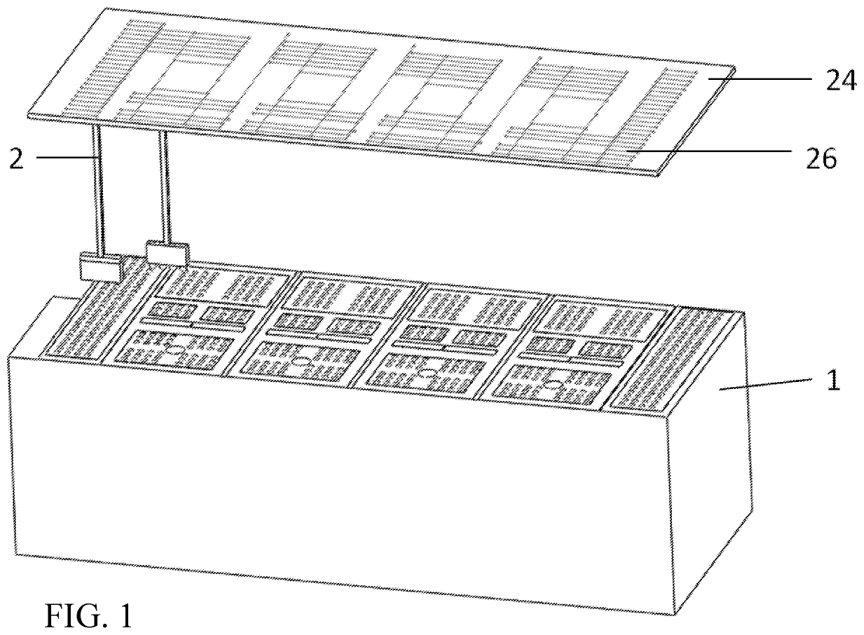

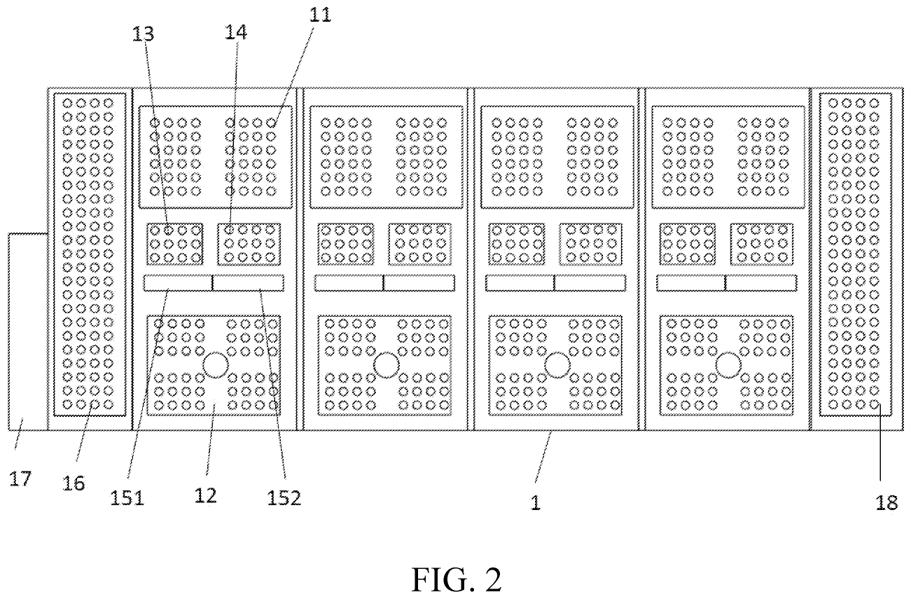

On the basis of Examples 1-2, the following embodiments are adjusted: Referring to FIGS. 1-2,

the entire enrichment process includes a plurality of oscillation incubation and centrifugation processes, each process including an oscillation incubation and centrifugation step. Therefore, an embodiment with a plurality of reaction areas is provided. Specifically, the reaction frame 1 of the whole apparatus is divided into a plurality of corresponding reaction areas according to the times of the oscillation incubation and centrifugation process. Each corresponding area is provided with an oscillation incubation structure 11, centrifugal structure 12, reagent placing structure 13, consumable placing structure 14, and waste placing structure 15. A set of operation frame 2 with a liquid taking and adding structure 22 and a tube body moving structure 21 is provided above each reaction area. In this way, a systematic process of liquid taking and tube body clamping can be realized without any conflicts in usage. The moving track 26 of the operation frame 2 is a track for facilitating movement of the operation frame 2 for liquid taking and tube body clamping. The moving track 26 is also divided into different areas to allow the moving track 26 to move in the current area and take samples or EP tubes in an up-moving area.

In the case only one batch of samples are enriched for one process, one reagent placing structure 13, one consumable placing structure 14, and one waste placing structure 15 are provided, and all the reagent placing structure, the consumable placing structure, and the waste placing structure for exosome enrichment are respectively and uniformly provided. In this way, the space occupied by the apparatus can be further reduced, and it takes less time to move the operation frame 2 since the overall structure is small.

A fault alarm structure 5 is further provided between each structure and the control structure 3, and when the corresponding fault alarm structure 5 sends an alarm signal, it indicates a malfunction in the corresponding structure. Or, the control structure 3 is further connected to a display structure, and the display structure displays the alarms from the corresponding alarm structure, prompting maintenance for the corresponding structure malfunction. This arrangement ensures that the alarm is given in a timely manner when some problems occur in this complex structure.

The number of reactions corresponding to the amount of any two different reagents in the reagent placing structure 13 is equal, and when the number of reactions reaches a certain threshold, a prompt or display on the display structure indicates the need for replacement. The number of reactions corresponding to the amount of any two different consumables in the consumable placing structure 14 is also equal, or the consumable amount of each consumable is equal to the number of reactions of the reagent, so as to facilitate overall replacement of reagents and consumables. The waste placing structure 15 is divided into a consumable discarding portion 151 and a liquid discarding portion 152, handling waste consumables and liquid, respectively. The capacity of the waste placing structure 15 for accommodating the waste is equal to the space required for accommodating reagents and consumables of one replacement, allowing it to be replaced together with the reagents and consumables. This ultimately reduces monitoring difficulties and increases efficiency.

The enriched sample collection structure 18 is provided with an EP tube consumable placing structure 14, or the EP tube consumable placing structure 14 is provided in another nearest reaction area; when proceeding to last step, the EP tube is moved to the enriched sample collection structure 18 zone through the tube body moving structure 21, then the supernatant obtained after enrichment is transferred to the EP tube through the liquid taking and adding structure 22 for collection, and then detection is performed.

### Example 4

Disclosed is a process for enriching neurogenic exosomes in blood using the apparatus according to Example 3, comprising: providing 4 reaction areas; placing the sample tube onto the sample placing structure 16, and moving, by the tube body moving structure 21, the sample tube into a first reaction area; performing at least one oscillation, incubation and centrifugation process, where the final step is the centrifugation process; adding the corresponding reagent when it is required; moving the sample tube or extracting the supernatant to the next reaction area to complete at least one oscillation, incubation and centrifugation process, adding the corresponding reagent in the process for a reaction, where the final step is the centrifugation process; when all the oscillation, incubation and centrifugation processes are finished, moving the sample tube or the supernatant to the final enriched sample collection structure 18 for subsequent detection.

The specific process is as follows:

The first transition operation includes: subjecting the sample tube to vortex oscillation and shaking incubation within the oscillation incubator, and then centrifuging the sample tube at a low temperature. This operation is intended to remove impure protein from the blood.

The second transition operation includes: subjecting the sample tube to vortex oscillation and standing incubation within the oscillation incubator, and then centrifuging the sample tube at a low temperature. This operation is intended to precipitate the total exosomes from plasma.

The third transition operation includes: subjecting the sample tube to vortex oscillation and low-temperature standing incubation within the oscillation incubator, then incubating the sample tube with a shaker at room temperature, and finally centrifuging the sample tube. This operation is intended to precipitate the neurogenic exosomes.

The fourth transition operation includes: subjecting the sample tube to vortex oscillation and room-temperature standing incubation within the oscillation incubator, and then centrifuging the sample tube at a low temperature. This operation is intended to lyse and re-suspend the neurogenic exosomes.

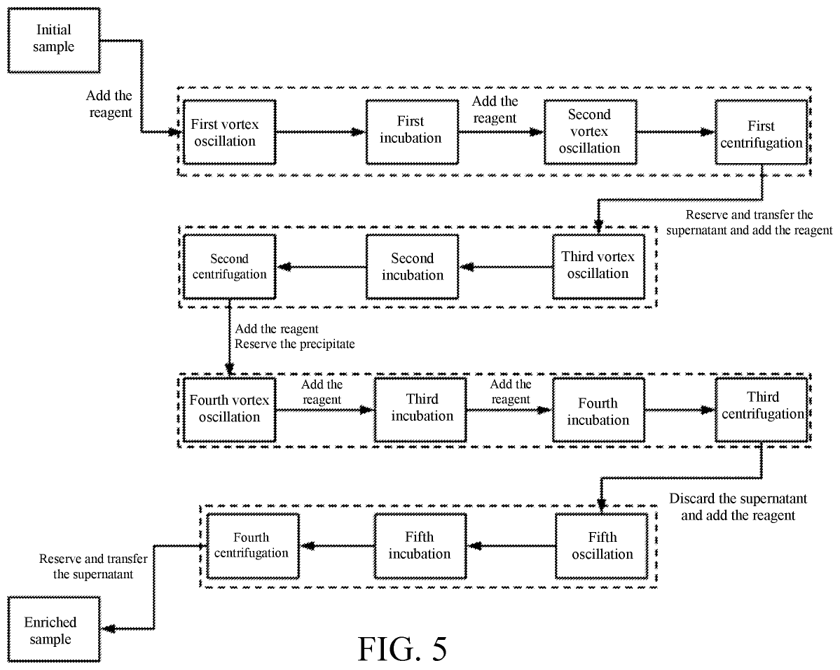

Referring to FIG. 5, only a schematic flow chart is shown in FIG. 5, which involves various steps for exosome enrichment.

The more specific steps are as follows:

The sample tubes loaded with samples are placed in sequence on the sample rack and loaded onto the machine.

The first transition operation includes:
1.1 adding reagent 1 into each sample tube, and performing vortex oscillation;
1.2. performing incubation with a shaker at room temperature;
1.3. adding reagent 2 and reagent 3 respectively, and performing vortex oscillation;
1.4. centrifuging at 4-8° C.,
where the centrifugal force was 1500-3000 g, the shaking amplitude was 300-800 rpm, and the amplitude of vortex oscillation was 1000-2000 rpm; the steps of the first transition operation were intended to remove impure protein from the blood.

The second transition operation includes:
2.1. transferring the supernatant to a new EP tube;
2.2. adding reagent 4, and performing vortex oscillation;
2.3. performing standing incubation at 4-8° C.;
2.4. centrifuging at 4-8° C., and discarding the supernatant 2 times,
where standing incubation was performed at 4° C., the centrifugal force was 1500-3000 g, and the amplitude of vortex oscillation was 1000-2000 rpm; the steps of the second transition operation were intended to precipitate the total exosomes from plasma.

The third transition operation includes:
3.1. adding reagent 5 into the precipitate;
3.2. performing vortex oscillation, incubating at 4-8° C., and determining the dissolution;
3.3. repeating step 3.2 until complete dissolution of the precipitate is detected;
3.4. adding reagent 6, and performing incubation with a shaker at room temperature;
3.5. adding reagent 7, and performing incubation with a shaker at room temperature;
3.6. centrifuging at 4-8° C. and discarding the supernatant,
where in steps 3.1 to 3.3, the amplitude of vortex oscillation was 1000-2000 rpm, and the total exosomes were re-suspend; in the other steps of the third transition operation, incubation was performed at room temperature, the shaking amplitude was 300-800 rpm, the centrifugal force was 200-800 g, and neurogenic exosomes were precipitated.

The fourth transition operation includes:
4.1. adding reagent 8, and performing vortex oscillation;
4.2. performing standing incubation at room temperature;
4.3. centrifuging at 4-8° C., and transferring the supernatant to a new EP tube,
where incubation was performed at room temperature, the centrifugal force was 1500-3000 g, and neurogenic exosomes were lysed and re-suspended.

At this point, a process of enriching neurogenic exosomes in blood is completed.

### Example 5

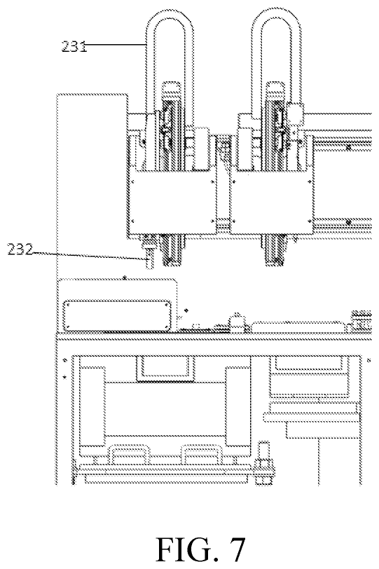

As shown in FIGS. 7-10, the specific embodiment provides an automated centrifugation system for exosome enrichment, including:
a centrifugal machine 12 including a sampling base 213 for holding a centrifuge tube 261,
a tube body moving structure 21 for clamping the centrifuge tube 261 and transporting the centrifuge tube 261 along a preset track, and
a sampling base identifier of the sampling base 213 configured to identify an identification feature of the sampling base 213 initially positioned at the identification station and the identification feature of the sampling base 213 subsequently positioned at the identification station, wherein, comparison is conducted to find out whether the identification feature of the sampling base 213 initially positioned at the identification station is the same as the identification feature of the sampling base 213 subsequently positioned at the identification station, if yes, stop the identification; if not, continue to identify the identification feature of the subsequent sampling base 213, and compare the identification feature with the identification feature of the initial sampling base 213.

In some application scenarios, such as in an exosome enrichment device (see Patent No. 2022108749840, Exosome Enrichment System), a preset track is a track associated with a mechanical arm 231.

The sampling base identifier in the present application may be any sampling base identifier that can indicate that all sampling bases 213 are placed into or removed from the centrifuge tube 261, for example, the sampling base identifier may be a magnetic sensor which has an operation logic substantially identical to the operating logic of the color identification sensor 241. The present implementation specifies the operation logic of the color identification sensor 241.

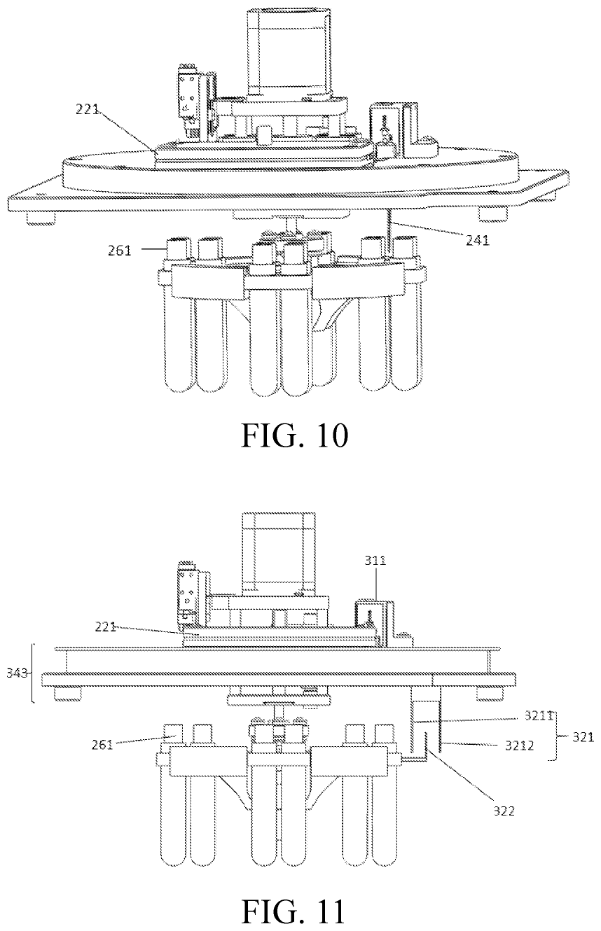

In some examples of the present application, the sampling base identifier is a color identification sensor 241, with the color as the identification feature; the sampling base 213 is provided with identification structures 251 that can be identified by the color identification sensor 241, each identification structure 251 having a different color. See FIGS. 10-11.

The working principle of this example is as follows: After the sampling base identifier of the sampling base 213 is started, an identification feature of the first sampling base 213 positioned at the identification station is identified, the identification feature of each subsequent sampling base 213 positioned at the identification station is identified by the sampling base identifier of the sampling base 213 and compared with the identification feature of the first sampling base 213 positioned at the identification station. If the identification feature identified subsequently is the same as the identification feature of the first sampling base 213, it indicates that all the sampling bases 213 have been identified, and identification is stopped; if the identification feature identified subsequently is different from the identification feature of the first sampling base 213, it indicates that there are still unidentified sampling bases 213, and identification is continued.

In the sampling state, the tube body moving structure 21 clamps the centrifuge tube 261 and transports the centrifuge tube 261 to the sampling base 213 along a preset track, and the sampling base 213 is positioned at the sampling station; in the extraction state, the tube body moving structure 21 clamps the centrifuge tube 261 in the sampling base 213 and transports the centrifuge tube 261 along a preset track, so that the centrifuge tube 261 leaves the sampling base 213; the color identification sensor 241 identifies the identification feature of the sampling base 213 at the identification station. In the working process, the color identification sensor 241 begins to identify the first sampling base 213 positioned at the identification station after being started up. During the sampling, the tube body moving structure 21 clamps the centrifuge tube 261, and places the centrifuge tube 261 in the hole site 214 of the sampling base 213 on the sampling station; after each hole sites 214 of the sampling base 213 is placed with the centrifuge tube 261, the centrifugal machine 12 starts to rotate under a preset program, and the full sampling base 213 enters the identification station; the next sampling base 213 is positioned at the sampling station, the tube body moving structure 21 continues to clamp the centrifuge tube 261, and places the centrifuge tube 261 in the hole site 214 of the sampling base 213, and the color identification sensor 241 identifies the identification feature of the sampling base 213 currently positioned at the identification station and compares the identification feature with the identification feature of the first sampling base 213. During the extraction, the first sampling base 213 is positioned at the sampling station, the tube body moving structure 21 clamps the centrifuge tube 261 and removes the centrifuge tube 261 from the sampling base 213; after all the centrifuge tubes 261 in the sampling base 213 are removed, the centrifugal machine 12 starts to rotate under a preset program, and the empty sampling base 213 leaves the sampling station and enters the identification station; the next sampling base 213 is positioned at the sampling station, the tube body moving structure 21 continues to clamp the centrifuge tube 261 located in the sampling base 213 at the sampling station, and the color identification sensor 241 identifies the identification feature of the sampling base 213 currently positioned at the identification station and compares the identification feature with the identification feature of the first sampling base 213 during the extraction.

In a specific working state, in the sampling state, the tube body moving structure 21 clamps the centrifuge tube 261 and transports the centrifuge tube 261 to the sampling base 213 along a preset track, and the sampling base 213 is positioned at the sampling station; in the extraction state, the tube body moving structure 21 clamps the centrifuge tube 261 in the sampling base 213 and transports the centrifuge tube 261 along a preset track, so that the centrifuge tube 261 leaves the sampling base 213; the color identification sensor 241 identifies the identification feature of the sampling base 213 at the identification station.

The color identification sensor 241 is in connection with the centrifugal machine 12 for data communication; in the sampling state, the centrifugal machine 12 rotates one of the empty sampling bases 213 to the sampling station, the tube body moving structure 21 clamps the centrifuge tube 261 and transports the centrifuge tube 261 to the sampling base 213 on the sampling station along a preset track; when the sampling base 213 is full, the centrifugal machine 12 rotates according to a preset program, so that the full sampling base 213 leaves the sampling station and enters the identification station, and the color identification sensor 241 identifies that the identification feature of the sampling base 213 currently positioned at the identification station is A1; the next empty sampling base 213 arrives at the sampling station, the tube body moving structure 21 clamps the centrifuge tube 261 and transports the centrifuge tube 261 to the sampling base 213 currently positioned at the sampling station along a preset track until the sampling base is full; the full sampling base 213 leaves the sampling station and enters the identification station, and the color identification sensor 241 identifies that the identification feature of the sampling base 213 currently positioned at the identification station is B 1; the color identification sensor 241 compares A1 with B1; if A1=B1, identification is stopped, the tube body moving structure 21 stops transporting the centrifuge tube 261 to the sampling base 213 on the sampling station, and the centrifugal machine 12 starts to rotate and centrifuges the centrifuge tube 261; if A1≠B1, the above operation is repeated.

In some examples of the present application, the color identification sensor 241 is in connection with the centrifugal machine 12 for data communication; in the extraction state, the centrifugal machine 12 rotates the sampling base 213 to the sampling station, the tube body moving structure 21 clamps the centrifuge tube 261 and removes the centrifuge tube 261 from the sampling base 213 on the sampling station along a preset track; when the centrifuge tubes 261 at all the hole sites 214 are removed from the sampling base 213, the centrifugal machine 12 rotates according to a preset program, so that the empty sampling base 213 enters the identification station, and the color identification sensor 241 identifies that the identification feature of the sampling base 213 currently positioned at the identification station is M1; the next sampling base 213 loaded with the centrifuge tube 261 arrives at the sampling station, the tube body moving structure 21 clamps the centrifuge tube 261 and removes the centrifuge tube 261 from the sampling base 213 on the sampling station along a preset track, the empty sampling base 213 leaves the sampling station to enter the identification station, and the color identification sensor 241 identifies that the identification feature of the sampling base 213 currently positioned at the identification station is N1; the color identification sensor 241 compares M1 and N1, and if M1=N1, identification is stopped, the tube body moving structure 21 stops removing the centrifuge tube 261 from the sampling base 213 on the sampling station, and the centrifugal machine 12 stop rotating; if M1≠N1, the above operation is repeated.

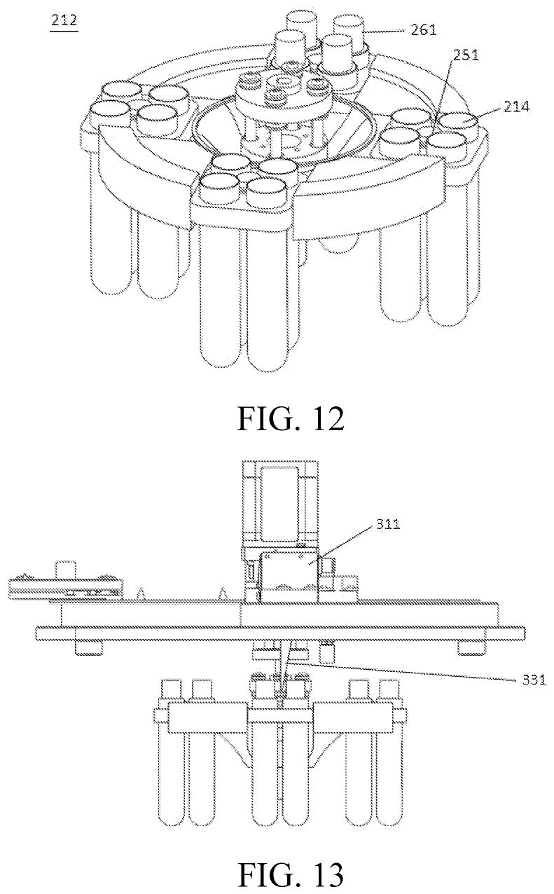

In some examples of the present application, the centrifugal machine 12 includes at least two sampling bases 213, the sampling bases 213 being symmetrical to each other. See FIG. 12.

Preferably, four sampling bases 213 are provided. The four sampling bases 213 are provided symmetrically with respect to each other, so as to ensure stability during the rotation of the centrifugal machine 12.

The sampling base 213 includes a hole site 214 for placing a centrifuge tube 261; furthermore, a buffer layer is provided on the inner wall of the hole site 214 and is fixedly connected to the inner wall of the hole site 214 to prevent the centrifuge tube 261 from colliding with the inner wall of the hole site 214 in the centrifugation process, thereby avoiding damage to the centrifuge tube 261.

Preferably, four hole sites 214 are provided on each sampling base 213; and the four hole sites 214 are disposed symmetrical to each other.

Further, the identification structure 251 is provided in the center of the sampling base 213. The identification structures 251 include a red identification structure 251, a yellow identification structure 251, a green identification structure 251, and a blue identification structure 251. Certainly, the identification structures 251 may be of any color capable of being identified by the color identification sensor 241. Specifically, the identification structure 251 is a blocky structure which may be a cylindrical blocky structure, a quadrilateral blocky structure, a triangular blocky structure, a polygonal blocky structure and an irregular blocky structure. Alternatively, the identification structure 251 is a columnar structure which may be a cylindrical structure, a triangular prism-shaped structure, a quadrangular prism-shaped structure, or a polygonal prism-shaped structure. That is, the identification structure 251 may be of an arbitrary shape and have a color that can be identified by the color identification sensor 241.

In some examples of the present application, the centrifugal machine 12 includes a centrifuge chamber 212, a drive structure, and a control structure 3. The drive structure drives the centrifuge chamber 212 to rotate, and the control structure 3 is in connection with the color identification sensor 241 for data communication, and the control structure 3 is in connection with the drive structure for data communication. The control structure 3 includes a preset program according to which the centrifugal machine 12 is rotated.

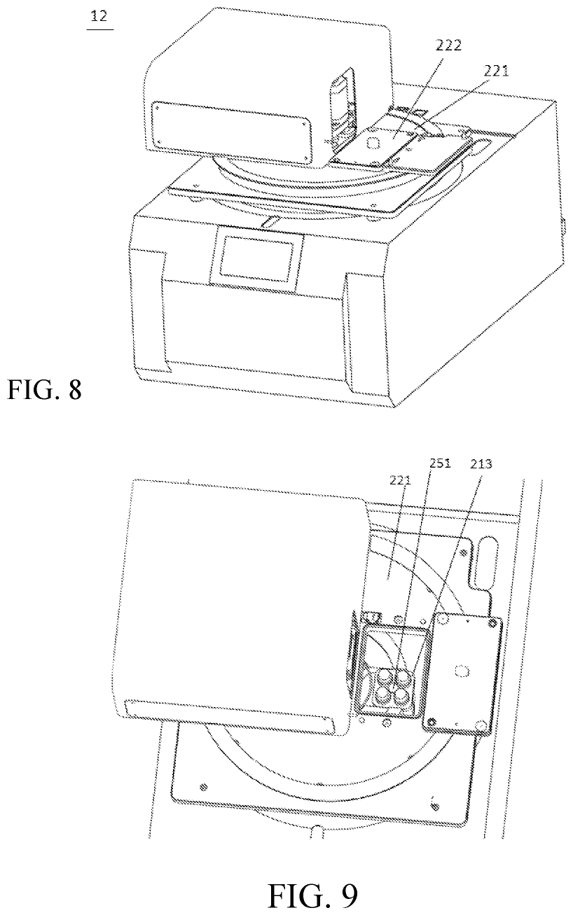

In some examples of the present application, the centrifugal machine 12 includes a dust cover 221, the dust cover 221 is located above the centrifuge chamber 212, and the dust cover 221 covers the centrifuge chamber 212. See FIGS. 7-8.

Specifically, an opening is formed on the dust cover 221 and corresponds to the position of the sampling station, and the tube body moving structure 21 transports the centrifuge tube 261 to the sampling base 213 through the opening or removes the centrifuge tube 261 from the sampling base 213.

Further, a cover 222 for closing the opening is provided, and the cover 222 is detachably connected to the dust cover 221. Specifically, a slot is formed at the bottom of the cover 222, and an upwardly protruding insertion plate is provided at the opening end of the dust cover 221 and is inserted into the slot, so that the cover 222 and the dust cover 221 are detachably connected to each other. In some examples of the present application, the tube body moving structure 21 includes a mechanical arm 231 and a clamping jaw 232, the clamping jaw 232 is fixedly connected to the mechanical arm 231, and the clamping jaw 232 clamps the centrifuge tube 261; the tube body moving structure 21 further includes a controller
311 and a driver, and the driver drives the mechanical arm 231 to move along a preset track, the clamping jaw 232 and the mechanical arm 231 moves synchronously, and the controller
311 includes a preset program which causes the mechanical arm 231 to move along a preset track, where the preset track cooperates with the mechanical arm 231 to enable the mechanical arm 231 to move.

### Example 6

A sample identification system for a centrifuge device in an exosome enrichment system is provided, including a sampling base identifier detachably secured to the centrifugal machine 12, the sampling base identifier being located above the centrifuge chamber 212 of the centrifugal machine 12; an identification structure 251 which can be identified by a sampling base identifier is provided on the centrifuge chamber 212, and the identification structure 251 is detachably connected to the centrifuge chamber 212; the sampling base identifier identifies the identification feature of the identification structure 251, and when the identification feature of the identification structure 251 is identified twice by the sampling base identifier, it indicates that the centrifuge chamber 212 has already completed one revolution, and the centrifugal machine 12 starts to enter the working or standby state.

The controller
311 is connected to the sampling base identifier by a wire, where the sampling base identifier sends an identification signal for identifying the identification structure 251 to the controller 311, and the controller
311 receives and processes the identification signal; the controller
311 is in connection with the centrifugal machine 12 for data communication.

As shown in FIGS. 11-13, 14, and 15, the sample identification system is used for an exosome enrichment device. In this application scenario, the centrifugal machine 12 includes a dust cover 221, a centrifuge chamber 212, and an outer housing, and the dust cover 221 is communicated with the centrifuge chamber 212, and the dust cover 221 is fixed on an operation platform of the exosome enrichment device; the interior of the exosome enrichment device includes a fixing component for supporting the centrifugal machine 12, where the fixing component provides support at the bottom of the centrifugal machine 12; the centrifuge chamber 212 includes a sampling base 213 for placing the centrifuge tube 261, and the number of sampling bases 213 is configured according to actual demands.

In some examples of the present application, the identification structure 251 is provided on the upper end surface of the centrifuge chamber 212, and the sampling base identifier is provided above the identification structure 251, or the sampling base identifier is provided on the side of the identification structure 251.

Specifically, in some examples of the present application, the sampling base identifier is a position sensor. The identification feature is the location of the identification structure 251.

In the application scenario of the exosome enrichment device, the controller
311 is provided on the upper end surface of the dust cover 221 of the centrifugal machine 12, and the position sensor and a controller are
311 connected by a wire. The exosome enrichment device includes a power supply structure, the controller
311 is connected to the power supply structure by a wire, and the sampling base identifier is connected to the power supply structure by a wire. The dust cover 221 is provided with a through hole, with one end of the position sensor fixedly connected to the dust cover 221 and the other end of the position sensor capable of passing through the through hole; the identification structure 251 is provided on the upper end surface of the centrifuge chamber 212, the identification structure 251 is fixedly connected to the centrifuge chamber 212, the initial positions of the identification structure 251 and the position sensor are on the same straight line perpendicular to the ground, the identification structure 251 and the centrifuge chamber 212 rotate synchronously, and the position sensor senses the position of the identification structure 251.

Specifically, the position sensor senses the position of the identification structure 251 and generates a position signal A, and the controller
311 acquires the position signal A; when the position sensor senses the position of the identification structure 251 again, a position signal B is generated, and the controller
311 acquires the position signal B, where when A=B, it indicates that the centrifuge chamber 212 has already completed one revolution, and at this time, if the centrifuge chamber 212 is already filled with the centrifuge tubes 261, the centrifugal machine 12 starts to rotate for centrifugation, and if there is no centrifuge tube 261 in the centrifuge chamber 212, it indicates that all the centrifuge tubes 261 after centrifugation have been removed, and the centrifugal machine 12 enters a standby state.

Alternatively, the identification structure 251 is provided on a side wall of the centrifuge chamber 212, the identification structure 251 is fixedly connected to the side wall of the centrifuge chamber 212, the initial positions of the identification structure 251 and the position sensor are on the same straight line parallel to the ground, the identification structure 251 and the centrifuge chamber 212 rotate synchronously, and the position sensor senses the position of the identification structure 251.

The position sensor can be any kind of sensor capable of realizing position sensing. As shown in FIGS. 1-2, in this example, the position sensor is an infrared sensor 321. The infrared sensor 321 is fixedly connected to the dust cover 221 of the centrifugal machine 12, and the infrared sensor 321 includes an emitting component 3211 and a receiving component 3212, where the emitting component 3211 and the receiving component 3212 are provided opposite to each other, and the emitting component 3211 emits an infrared light beam which is received by the receiving component 3212, thereby forming a line formed by the infrared light beam. The identification structure 251 is a light shielding plate 322; the light shielding plate 322 is fixedly connected to the side wall of the centrifuge chamber 212, and the initial position of the light shielding plate 322 is between the emitting component 3211 and the receiving component 3212. The light shielding plate 322 cuts off the line formed by the infrared light beam, the centrifuge chamber 212 rotates to enable the light shielding plate 322 to rotate, and the light shielding plate 322 leaves the initial position, and when the light shielding plate 322 cuts off the line formed by the infrared light beams again, it indicates that the centrifuge chamber 212 has already completed one revolution, and the centrifugal machine 12 starts to enter the working or standby state.

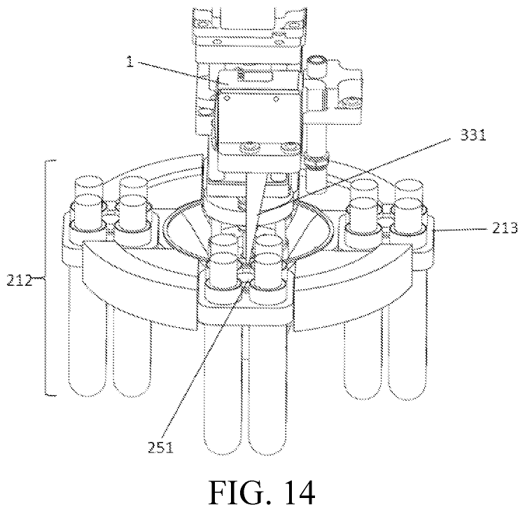

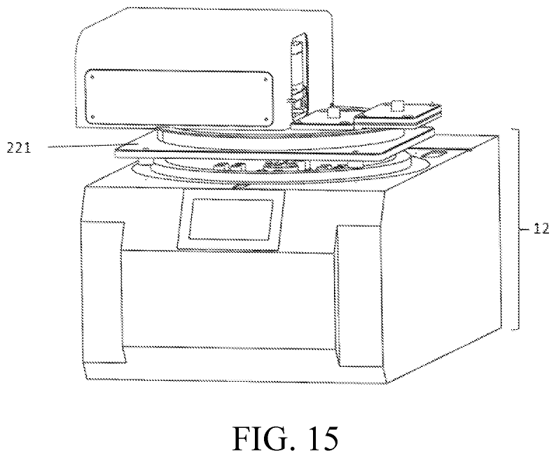

As shown in FIGS. 14-15, in another example, the sampling base identifier is a color identification sensor 241.

Correspondingly, the identification feature is the color of the identification structure 251.

An identification structure 251 having a color recognizable by the color identification sensor 241 is provided at the upper end of the identification structure 251, and the identification structure 251 is fixedly connected to the identification structure 251. The identification structure 251 is provided on the upper end surface of the centrifuge chamber 212, specifically, the identification structure 251 is a blocky structure, and the identification structure 251 and the centrifuge chamber 212 are connected by screws. Certainly, the identification structure 251 and the centrifuge chamber 212 may be connected through any other means capable of achieving a relative fixed position between them, not limited to the method provided in this example.

The number of the identification structures 251 is the same as that of the sampling bases 213 in the centrifugal machine 12. In some application scenarios, if 4 sampling bases 213 are provided in the centrifugal machine 12, 4 identification structures 251 are provided, the identification structures 251 are provided in the center of the sampling bases 213, and an identification structure 251 with a different color is provided at the upper end of each identification structure 251. When the identification structure 251 and the color identification sensor 241 are on the same straight line perpendicular to the ground, the identification structure 251 is identified by the color identification sensor 241.

The color identification sensor 241 identifies the color of each of the identification structures 251, and when the color of one of the identification structures 251 is identified again, it indicates that the centrifuge chamber 212 has already completed one revolution. The centrifugal machine 12 then starts to enter the working or standby state.

The description of the above examples is only intended for the understanding of the present invention. It should be noted that for those of ordinary skill in the art, without departing from the principle of the present invention, several improvements can also be made to the present invention, and these improvements also fall within the protection scope of the claims of the present invention.

What is claimed is:

1. An automated apparatus for effectively enriching neurogenic exosomes in blood, comprising:
an oscillation incubator for performing oscillation incubation during enrichment of neurogenic exosomes in blood,
a centrifugal machine for performing centrifugation during enrichment of neurogenic exosomes in blood, and
a control structure for controlling transition of a sample tube between the oscillation incubator and the centrifugal machine according to a process of enriching neurogenic exosomes in blood.

2. The automated apparatus of claim 1, wherein the control structure controls the transition of the sample tube between the oscillation incubator and the centrifugal machine, wherein a transfer from one centrifugation operation to a next oscillation incubation operation is counted as one transition, and a process starting from the beginning of an oscillation incubation operation until the beginning of a next oscillation incubation operation after undergoing at least one centrifugation operation is defined as one transition operation; one transition operation comprises at least one centrifugation action and at least one oscillation incubation action.

3. The automated apparatus of claim 2, wherein one transition operation is used to precipitate the neurogenic exosomes and one transition operation is used to lyse and re-suspend the neurogenic exosomes.

4. The automated apparatus of claim 1, comprising,
a reaction frame provided at a bottom and used for placing a reactor for enriching neurogenic exosomes in blood, wherein
the reactor comprises at least the oscillation incubator and the centrifugal machine, and
an operation frame provided above the reaction frame, comprising:
a tube body moving structure for carrying a blood sample tube containing exosomes to move among different reactors,
a liquid taking and adding structure for taking and adding liquid from/to the blood sample tube in the process of enriching exosomes, and
a control structure for controlling working parameters and start and stop of the reactor and controlling the operation frame to move among different reactors, wherein
the liquid taking and adding structure is controlled by the control structure to move between the blood sample tube and a reagent placing structure to enable a liquid taking and adding action on the blood sample tube; the tube body moving structure is controlled by the control structure to carry the blood sample tube to transit between the oscillation incubator and the centrifugal machine.

5. The automated apparatus for effectively enriching neurogenic exosomes in blood of claim 4, wherein when it is required to take liquid into a new blood sample tube when exosomes are enriched, the new blood sample tube is first moved onto the reactor by the tube body moving structure, and then the required liquid is taken into the new sample tube by using the liquid taking and adding structure.

6. The automated apparatus for effectively enriching neurogenic exosomes in blood of claim 4, wherein enriching exosomes in blood requires 3-4 transition operations;
exosomes in blood are enriched in one batch at a time; a set of oscillation incubator and centrifugal machine are provided, and a plurality of transition operations are completed in the same set of oscillation incubator and centrifugal machine; or, an oscillation incubator and a centrifugal machine are provided according to a number of different types of sample tubes;
or, a plurality of sets of oscillation incubators and centrifugal machines are provided; a number of sets of oscillation incubators and centrifugal machines is the same as a number of transition operations in the whole process of enriching neurogenic exosomes in blood; neurogenic exosomes in blood are enriched in multiple batches according to the process.

7. The automated apparatus for effectively enriching neurogenic exosomes in blood of claim 6, further comprising a reagent placing structure for placing a reagent required for enrichment of neurogenic exosomes in blood, a consumable placing structure for placing consumables required for enrichment of neurogenic exosomes in blood,
and a waste placing structure, wherein a set of reagent placing structure, consumable placing structure and waste placing structure are provided on a placement rack as a whole; or a number of the reagent placing structures, the consumable placing structures and the waste placing structures is the same as that of the oscillation incubators or the centrifugal machines.

8. The automated apparatus for effectively enriching neurogenic exosomes in blood of claim 6, wherein the reaction frame is divided into reaction areas equal to a number of transition operations according to the number of transition operations during the whole process of enriching neurogenic exosomes in blood, and each reaction area is internally provided with a set of oscillation incubator and centrifugal machine.

9. The automated apparatus for effectively enriching neurogenic exosomes in blood of claim 4, wherein the operation frame is provided on an arrangement top; the arrangement top is provided above the reaction frame and maintains a fixed position relative to the reaction frame, and the arrangement top is provided with a moving track allowing the operation frame to move above any structure and perform a corresponding operation, or the arrangement top is provided with a three-dimensional moving mechanical arm for meeting mobility requirements of the operation frame.

10. The automated apparatus for effectively enriching neurogenic exosomes in blood of claim 4, wherein the operation frame is an integral frame, and the liquid taking and adding structure and the tube body moving structure are provided thereon; the liquid taking and adding structure and the tube body moving structure are capable of moving up and down on the operation frame, allowing a single structure to perform all functions.

11. The automated apparatus for effectively enriching neurogenic exosomes in blood of claim 4, wherein two operation frames are provided, namely a liquid taking and adding operation frame and a tube body moving operation frame; the operation frames move to positions above corresponding structures respectively for performing an action, and when one operation frame is in action, the other operation frame moves along a moving track to a position where the other operation frame will not be interfered with.

12. The automated apparatus for effectively enriching neurogenic exosomes in blood of claim 11, wherein the moving track of the operation frame is a track for facilitating movement of the operation frame for liquid taking and tube body clamping.

13. The automated apparatus for effectively enriching neurogenic exosomes in blood of claim 4, wherein the liquid taking and adding structure is provided with a same number of operating heads as the tube body moving structure.

14. The automated apparatus for effectively enriching neurogenic exosomes in blood of claim 13, wherein corresponding to a single type of reagent, the reagent placing structure is provided with liquid taking ports in a quantity equal to a number of the operating heads of the liquid taking and adding structure, and a positional relationship of the liquid taking ports corresponds to a positional relationship of the operating heads; corresponding to a single type of consumable, a consumable placing structure is provided with consumable taking ports in a quantity equal to a number of the operating heads of the liquid taking and adding structure, and a positional relationship of the consumable taking ports corresponds to a positional relationship of the operating heads.

15. The automated apparatus for effectively enriching neurogenic exosomes in blood of claim 4, wherein a number of reactions corresponding to an amount of any two different consumables in the consumable placing structure is also equal; or, a consumable amount of each consumable is equal to a number of reactions of a reagent.

16. The automated apparatus for effectively enriching neurogenic exosomes in blood of claim 4, comprising a centrifugation system, wherein the centrifugation system comprises a centrifugal machine comprising a sampling base for placing a centrifuge tube,
a tube body moving structure for clamping the centrifuge tube 6 and transporting the centrifuge tube along a preset track, and
a sampling base identifier configured to identify an identification feature of the sampling base initially positioned at an identification station and an identification feature of a sampling base subsequently positioned at the identification station, wherein, comparison is conducted to find out whether the identification feature of the sampling base initially positioned at the identification station is the same as the identification feature of the sampling base subsequently positioned at the identification station; if yes, stop the identification; if not, continue to identify an identification feature of a subsequent sampling base, and compare the identification feature with the identification feature of the initial sampling base.

17. The automated apparatus for effectively enriching neurogenic exosomes in blood of claim 16, wherein
the sampling base identifier is a color identification sensor, with a color as an identification feature; the sampling base is provided with identification structures that can be identified by the color identification sensor, each identification structure having a different color;
after the sampling base identifier is started, an identification feature of the first sampling base positioned at the identification station is identified, an identification feature of each subsequent sampling base positioned at the identification station is identified by the sampling base identifier and compared with the identification feature of the first sampling base positioned at the identification station; if the identification features are the same, stop the identification; if the identification features are different, continue the identification until the identification features are the same.

18. The automated apparatus for effectively enriching neurogenic exosomes in blood of claim 16, wherein the sampling base identifier is a position sensor, with the position of an identification structure as the identification feature; the position sensor senses the position of the identification structure and generates a position signal A, and a controller acquires the position signal A; when the position sensor senses the position of the identification structure again, a position signal B is generated, and the controller acquires the position signal B, where when A=B, identification is stopped.

19. The automated apparatus for effectively enriching neurogenic exosomes in blood of claim 18, wherein the identification structure is provided on an upper end surface of a centrifuge chamber, and the sampling base identifier is provided above the identification structure, or the sampling base identifier is provided on a side of the identification structure.

20. The automated apparatus for effectively enriching neurogenic exosomes in blood of claim 16, wherein the centrifugal machine comprises a dust cover and a centrifuge chamber, and the dust cover is located above the centrifuge chamber and covers the centrifuge chamber; an opening is formed on the dust cover, and the tube body moving structure transports the centrifuge tube to the sampling base through the opening or removes the centrifuge tube from the sampling base; a cover for closing the opening in a centrifugal state is provided.
